(19) United States
(12) Patent Application Publication
(10) Pub. No.: US 2010/0021392 A1
(43) Pub. Date: Jan. 28, 2010

(54) Vapor-phase application of spinosyn for the control of pest, and formulations and products utilizing the same

(76) Inventor: Christine Kritikou, Krifissia (GR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/990,995

(22) PCT Filed: May 24, 2007
(86) PCT No.: PCT/IB2007/003176

§ 371 Date: Aug. 21, 2009

Related U.S. Application Data
(60) Provisional application No. 60/803,308, filed on May 26, 2006.
(60) Provisional application No. 60/859,965, filed on Nov. 20, 2006.

(51) Int. Cl. A01N 25/00; A01N 43/02; A61L 2/22; A23L 3/34

(52) U.S. Cl. 424/40; 514/450; 422/28; 426/335

(57) ABSTRACT

The present invention relates to the vapor-phase application of at least one spinosyn to treat pests including, for example, adult mosquitoes and houseflies.

This application claims priority to U.S. Provisional Patent Application No. 60/803,308 filed on May 26, 2006, and U.S. Provisional Patent Application No. 60/859,965 filed on Nov. 20, 2006.

### DESCRIPTION OF THE INVENTION

Pests and other insects cost farmers billions of dollars annually in crop losses and in expenses associated with keeping these pests under control. The losses caused by pests in agricultural environments include decreases in crop yield, reduced crop quality, and increased harvesting costs. Additionally, mosquitoes, houseflies and other insects cost society in ways which cannot be reduced to a mere dollar amount, for example, over a million people die each year from mosquito-borne diseases. Malaria, transmitted by the Anopheles mosquito, is a leading cause of death and disease worldwide causing between 300 and 500 million cases of malaria per year, particularly in developing countries. Like mosquitoes, houseflies are recognized to carry disease and have been identified as a factor in the spread of the trachoma virus and its associated Haemophilus bacterium in, for example, India and North Africa.

Nuisance pests can be described as the insects and pests that one finds commonly around the home and garden. While these insects are not usually associated with the carrying of disease, they nonetheless provide a significant bother to humans and millions of dollars are spent annually to develop products to control these nuisance pests.

The success of insecticide-based control programs in reducing the prevalence of insect vector-borne diseases has been accompanied by a growing interest in the possible harmful effects of widescale and prolonged use of synthetic insecticides on human health and the environment. Mosquito resistance to a number of conventional chemical insecticides is also a matter of current concern. In the majority of cases, not only does resistance render the selected compound ineffective but it often also confers cross-resistance to other chemically related compounds as compounds within a specific chemical group usually share a common target site within the pest, and thus, share a common mode of action.

The control of insects and pests which bother and/or harm people, animals and crops has been the subject of extensive research and there are many well-known methods and products for killing or reducing the population of pests and insects. Nonetheless, new, safe, and effective methods, compositions, and delivery systems are needed to control these pest and insect populations.

One series of active ingredients that have been used in solid and liquid form, as pest controls are spinosyns. Spinosyns are known fermentation products derived from the naturally occurring bacteria Saccharopolyspora spinosa. The family of compounds derived from this bacteria are generally known as spinosyns and have been referred to as factors or components A, B, C, D, E, F, G, H, I, J, K, L, M, N, O, P, Q, R, S, T, U V, W, Y, and the like, as described in U.S. Pat. Nos. 5,362,634, and 6,821,526 and published applications WO 93/09126 and WO 94/20518 which are each incorporated herein by reference in their entirety.

Each spinosyn has a 12-membered macrocyclic ring that is part of a tetracyclic ring system to which two different sugars are attached, the amino-sugar forosamine and the neutral sugar 2N,3N,4N-tri-O-methylrhamnose.

As used herein, the term “spinosyn” refers to a class of compounds which are based upon the fermentation products from the naturally occurring bacteria, Saccharopolyspora spinosa (species and subspecies) or a biologically modified form of this bacteria. The class of compounds known as spinosyns includes naturally occurring fermentation products, any after developed chemical source of those products, and any chemical modifications thereof.

The term “spinosyn” refers to an individual spinosyn, or a physiologically acceptable derivative or salt thereof, or a combination thereof and all isomers of the compounds. In addition as used herein the term “spinosyn” includes the semi-synthetic spinosyn analogues as described in U.S. Pat. No. 6,001,981, in which the chemically accessible areas of the spinosyn molecule were successfully substituted in a variety of ways. The term “spinosyn” also includes the novel biologically-active compounds as described in U.S. Patent No. 2006/0040877 produced by methods of using the hybrid polyketide synthase DNA to change the products made by spinosyn producing strains. Further, the term “spinosyn” includes the next generation spinosyns, e.g., XDE-175 having the tradename SPINETORAM, and S-1947. As defined in published U.S. patent application No. 2007/0104750, Spinetoram is a mixture of (2R,3aR,5aR,5bS,9S,13S,14R,16aS,16bR)-13-{[(2R,5S,6R)-5-(dimethylamino)-6-methyltetrahydro-2H-pyran-2-yl]oxy}-9-ethyl-14-methyl-7,15-dioxo-2,3,3a,4-,5,5a,5b,6,7,9,10,11,12,13,14,15,16a,16b-octadecahydro-1H-as-indaceno[3,2-d]oxacyclododecin-2-yl 6-deoxy-3-O-ethyl-2,4-di-O-methyl-.alpha.-L-mannopyranoside [DE-175J] and (2S,3aR,5aS,5bS,9S,13S,14R,16aS,16bS)-13-{[(2R,5S,6R)-5-(dimethylamino)-6-methyltetrahydro-2H-pyran-2-yl]oxy}-9-ethyl-4,14-dimethyl-7,15-dioxo-2,3,-3a,5a,5b,6,7,9,10,11,12,13,14,15,16a,16b-hexadecahydro-1H-as-indaceno[3,2-d]oxacyclododecin-2-yl 6-deoxy-3-O-ethyl-2,4-di-O-methyl-.alpha.-L-mannopyranoside in the proportion 50-90% to 50-10%.

Finally, the term “spinosyn” includes new spinosyn derivatives which may have a different spectrum of insecticidal activity produced using the cloned Saccharopolyspora spinosa DNA as described in U.S. Pat. No. 7,015,001. Different patterns of control may be provided by biosynthetic intermediates of the spinosyns or by their derivatives produced in vivo, or by derivatives resulting from their chemical modification in vitro. Such biosynthetic intermediates of the spinosyns are considered to belong to the class of “spinosyns” as described herein for use in the present invention.

Additionally the term “spinosyn or a derivative thereof” as used herein also refers to an individual spinosyn factor (A, B, C, D, E, F, G, H, J, K, L, M, N, O, P, Q, R, S, T, U, V, W, or Y), an N-demethyl derivative of an individual spinosyn factor, or a combination thereof or spinosyn aglycone compounds, i.e., compounds which have the macrolide backbone of the spinosyns but no sugar radicals.

Spinosyn A and spinosyn D are two spinosyns that have been found to be particularly useful active ingredients in pest and insect control formulations. A product comprised mainly of these two spinosyns, for example, in a ratio of approximately 85% spinosyn A to about 15% spinosyn D, is known as Spinosad and is produced by Dow AgroSciences (Indianapolis, Ind.). Spinosad is an active ingredient in several insecticide formulations available commercially from Dow AgroSciences, including, for example, those marketed under the tradenames TRACER, SUCCESS, SPINTOR, LASER, and ENTRUST. The TRACER product, for example, is comprised of about 44% to about 48% spinosad (w/v), while ENTRUST is a white to off-white solid powder containing about 80% spinosad.

Spinosad has been shown to be highly effective in the control of pests and insects including, but not limited to species from the orders Lepidoptera, Diptera, Hymenoptera, Thysanoptera, and a few Coleoptera. In addition, formulations comprising Spinosad have been shown to be highly effective in a variety of different environments including agricultural, horticultural, greenhouses, golf courses, gardens, homes, and the like. As used herein, the terms “effective,” “effective in the control of,” and “effective for control” or “control” are all used interchangeably and all refer to the ability of the composition/active to act upon the pest. As used herein, a product is considered effective if it reduces the effect of the pest on the surrounding environment. The desired effect of the pest on its surroundings that one wishes to control can vary. Non-limiting examples of the desired effects include, a reduction in biting, a reduction in the population, sterilization which can ultimately result in a reduction in population, suppressing insect development (insect growth regulators), cessation of feeding, reduced mobility, lack of orientation, repellency, knock down effect and mortality.

Spinosyns including Spinosad have been found to have very favorable environmental and toxicological profiles, thus making them particularly advantageous in the control of pests and insects. Spinosyns, and Spinosad in particular, have been found to be effective at lower use rates that other common insecticide and pesticide actives. In addition, Spinosad has been found to be highly effective in the control of detrimental pests and insects while maintaining minimum disruption to beneficial insects and other non-target organisms. Moreover, Spinosad has been shown to be relatively low in toxicity to mammals and birds and is only slightly toxic to fish. Chronic toxicology tests in mammals have shown that Spinosad is not carcinogenic, teratogenic, mutagenic, or neurotoxic.

The present invention relates to a vapor-phase application of spinosyns and spinosyn formulations for controlling pests and insects. According to one embodiment, the present invention relates to the vapor-phase application of a composition comprising Spinosad. Applying an insecticide or pesticide in the vapor-phase can provide advantages which would be readily apparent to the skilled artisan depending upon the pest or insect to be treated and the area in which the product is to be applied. By way of example only, a vapor-phase product may permeate into spaces that might not be easily reached by, for example, an applied spray or bait. Further, vapor-phase applications may target pests or insects and/or provide better or extended coverage over areas that may not be easily treated by conventional sprays and contact pesticides and insecticides.

It has been found that at least one spinosyn, and according to one embodiment, Spinosad, can be applied in the vapor-phase as an effective pest and insect control. As used herein “pest” and “pesticide” refer to the general class of organisms which have characteristics that are regarded as injurious or unwanted or which cause detrimental effects. Pesticides are recognized to include, for example, insecticides, fungicides and herbicides. By contrast “insect” and “insecticide” refer only to one subgroup of pests and pesticides. The vapor-phase application process according to the present invention can be used in both agricultural, i.e., pesticide/crop applications (e.g., thermal foggers where the pesticide is diluted with kerosene or another art recognized diluent and vaporized with heat into a dense fog, which allows tracking the plume downwind to target areas) and also in more general household or insecticide applications or in public health applications for example the control of mosquito species of medical importance like anopheles (e.g., the spinosyn could be incorporated in a net and be vaporized with the help of heat from a small resistance).

According to one embodiment, it has been found that these vapor-phase formulations are particularly suitable for the control of pests under conditions, which may be well served by a vaporized product, for example, in the protection of agricultural crops. A non-exhaustive list of crops for which spinosad is a useful pest control agent includes cotton, crucifers, leafy vegetables, citrus, apples, pears, stone fruit, tobacco, almonds, corn, wheat and cereals, potatoes, tomatoes, peppers, tropical tree fruits, turf and ornamentals, legume vegetables, grapes, soybean, rice, cucurbits, home and gardens, and the like. According to one embodiment, for example, the vapor phase spinosyn is used to control several hard-to-manage pests in stored grains. Using this novel spinosyn application one may achieve reduced use rates and limited contact of the grains with the active.

According to another embodiment, it has been found that the vapor-phase application of at least one spinosyn, for example, Spinosad, is suitable for the control of flying insects and pests, for example, adult mosquitoes and houseflies. While spinosyns have heretofore been known to be effective when ingested by pests thereby causing rapid excitation of the nervous system, these applications have required the ingestion of the active ingredient by the target pest or insect. Therefore, these actives have mainly been used in bait station devices as described in U.S. Pat. Nos. 6,821,562 B1 and 6,6,585,990 B1, both of which are incorporated herein by reference. In addition, spinosyns have also been used in formulations topically applied to animals to treat for infestations as described in U.S. Pat. No. 6,933,318 B1, which is incorporated herein by reference. Spinosyns have been used for treating the soil, for treating seeds or plant propagation materials, and for drenching and irrigating plants, as described in U.S. Pat. No. 6,583,088 B1, which is also incorporated herein by reference. Finally, spinosyns have been used in an aqueous base for the treatment of mosquitoes and bollworms. Due to the low vapor pressures of spinosyn A and D (2.4 and 1.6×10⁻¹⁰ mmHg at 25° C., respectively), Spinosad has generally been considered non-volatile and thus, the application of this product in the vapor-phase has not been readily apparent to the skilled artisan. The present invention demonstrates that spinosyns can be brought into the vapor phase for the control of insects, including flying insects. The vapor-phase spinosyn compositions of the present invention may be used in place of these prior direct contact/ingestion applications.

As used herein the term vapor-phase refers to the condition of the material or formulation when it is a vapor, i.e., rendered in a gaseous state, by any means, but specifically including the application of heat to render the spinosyn material in the gas phase. While vaporization refers to any method for rendering the material in a vapor phase, the term evaporation refers to vaporization below the boiling point of the material/formulation.

Not wishing to be bound by theory, the present inventor discovered that spinosyn containing compositions may be evaporated under appropriate conditions and are effective in the vapor-phase to repel and/or prevent biting by and/or knock down of and/or kill of target insects and pests either through direct contact with the vapor or by inhalation of the vapor by the target pest or insect. According to one embodiment, the formulations comprising at least one spinosyn are combined with a vehicle to cause evaporation. Vehicles to cause evaporation of the at least one spinosyn include one or more of, for example, heat, vacuum pressure, forced airflow (ventilation), combustion, and solvents.

The primary active ingredient for use in the present invention comprises at least one spinosyn from the class of spinosyns as described above. The active ingredient should be present in an amount sufficient to be active as against the pest or insect targeted. The upper limit of activity may be driven by characteristics of cost and toxicity that would be readily apparent to the skilled artisan. One skilled in the art would recognize that the amount of spinosyn could be reduced in the event a second active were present, so long as the combined composition is active as against the target pest or insect.

According to one embodiment, the spinosyn is present in the composition in an amount in the range from 0.1% and 90%. According to another embodiment, the spinosyn is present in an amount in the range from 0.1% and 15%. According to yet another embodiment, the spinosyn is present in an amount in the range from 0.5% to 10%, for example from 1% to 10%. Still further, the spinosyn may be present in an amount in the range of 2% to 10%. When the spinosyn is applied in the vapor-phase from a mat as will be described below, the spinosyn is present on the mat in an amount of from 1% to 10%.

According to one embodiment, heat is used as a vaporizing vehicle. The amount of heat applied will depend upon the nature of the particular spinosyn formulation to be evaporated. Generally, spinosyns have a very low vapor pressure and thus, spinosyns may be combined with solvents that carry the spinosyn along into the vapor phase making it easier to evaporate the spinosyn using an appropriate amount of heat. It will be understood that heat alone may be used to vaporize spinosyn according to the present invention and further a solvent may be present but may not act to dissolve the spinosyn or to modify its evaporation profile but rather may be, for example, merely assist with the physical distribution of the spinosyn upon an appropriate substrate. After selection of an appropriate solvent, selection of the appropriate amount of heat necessary to evaporate the composition will be readily apparent to the skilled artisan.

According to one embodiment, solvents for use in the present invention include any art recognized solvent for a spinosyn which allow the active to be fully or partially dissolved, which help the active to be spread on a storage medium or which will allow the spinosyn to evaporate with the application of heat. Characteristics for consideration in selecting an appropriate solvent include the pest to be treated, the desired product for applying the vapor phase spinosyn, the physicochemical properties of the specific spinosyn and the environment in which the product will be applied. As would be apparent to the skilled artisan, solvents for use in the present invention are preferably not carcinogenic and not classified as a dangerous or very dangerous chemical substance, particularly when the product is intended for household use. Other characteristics that would generally be considered include, heavy odors, which could be unpleasant and unacceptable to consumers, flammability and flash point depending upon the amount and type of heat to be used, and evaporation rate.

A non-exhaustive list of solvents for use with spinosyns includes water; ketones, for example, acetone, butanone, or methyl isobutyl ketone; nitrites, for example, acetonitrile; acetates, for example, ethyl acetate and amyl acetate; aromatic hydrocarbons for example toluene; isoparaffins and aliphatics hydrocarbons, for example shellsol; alcohols, for example, methanol, ethanol, benzyl alcohol, and isopropanol; chlorinated hydrocarbons for example, CH₂Cl₂, ethers, for example, ethylene glycol phenyl ether, esters, for example, isopropyl myristate, surfactants, oils, xylenes, acetylenics, and organic acids.

According to one embodiment of the present invention, ethyl alcohol is used as the solvent. According to another embodiment of the present invention, ethylene glycol phenyl ether is used as the solvent. According to yet another embodiment methyl isobutyl ketone may be used as the solvent.

The solvent may be present in an amount effective to dissolve the spinosyn, allow the active spinosyn to be spread on a storage medium (e.g., a mat paper) and/or to evaporate with the application of heat. According to one embodiment, the solvent is present in an amount ranging from 10% to 99.9%. According to another embodiment, the solvent is present in an amount ranging from 85% to 99.9%. According to yet another embodiment, the solvent is present in an amount of from 90% to 99%. One skilled in the art would clearly recognize that when a combination of vaporizing vehicles are applied, for example, heat or vacuum pressure is applied to a solvated spinosyn, the amount of heat or pressure necessary will be adjusted based upon the amount and type of the solvent and the chemical and physical characteristics of the specific spinosyn.

Formulations for use with the present invention may also contain suitable secondary active ingredients. A secondary active ingredient is any compound which itself is active against the target pest or which aides or assist the primary active against the target pest. Non-limiting examples of such secondary active ingredients include pyrethroids (as an example allethrin, transfluthrin), nicotinoids (as an example imidacloprid), fiproles (as an example fipronil), organophosphates (as an example malathion), carbamates (as an example carbaryl), botanicals (as an example pyrethrum, d-limonene), fumigants (as an example sulfuryl fluoride), and hydramethylnon. Appropriate combinations will be readily apparent to the skilled artisan. Such combinations could take advantage of different physical and biological properties of more than one active together in a single product. They could likewise be used for example, in a product aimed at treating more than one pest with a single application or in a product aimed at achieving for example both repellent/killing or knockdown/killing results at the same time.

Formulations for use in the present invention can also include non-active ingredients commonly associated with pesticides and insecticides, including, but not limited to perfumes, colorants, stabilizers, anti-oxidants, retardants, evaporation accelerating agents, synergists, emulsifiers, surfactants, silicon compounds and attractants.

Common non-active ingredients for use with the present invention include piperonyl butoxide, MGK, Verbutin, butyl-hydroxy-toluol (BHT), and the like. Piperonyl butoxide may be a non-reactive ingredient but in certain compositions it may play the role of a secondary active ingredient, enhancing the activity of the primary active agent or alternatively it may act as an evaporation retardant. Other art recognized materials typically included in products of this nature will be readily apparent to one of ordinary skill in the art.

The present invention further relates to products that may be produced for vapor phase application utilizing the vaporizable formulation as described herein. The product may take any art recognized or after developed form that can be used with a vapor-phase active ingredient.

According to one embodiment, the formulation for vaporization may be impregnated into a pad or mat. When the mat or pad is heated by a diffuser, the formulation is evaporated. Pads or mats for use in the pest control products of the present invention may be selected for their ability to absorb as much formulation as possible and for allowing, when heated, the formulation to be effectively evaporated. Appropriate mat configurations can be chosen from any art recognized mats (made, for example, from paper, plastic, fabric, ceramic materials or some combination thereof) and will be readily apparent to the skilled artisan.

According to one embodiment of the present invention, the pads or mats may be produced from paper. Appropriate thickness for the mats would be readily understood by the skilled artisan. According to one embodiment, the paper mats may have a thickness in the range of from 0.1 mm to 10 mm. Paper mats were evaluated at thicknesses of 2.5 mm, 2.7 mm, and 3.2 mm and were all found to work satisfactorily. According to one embodiment, a paper mat with a thickness of 2.5 mm was used. Other attributes which may be considered when selecting a paper mat include weight, break length, moisture content, surface pH and water capillary rise. According to one embodiment, the paper mat had the following characteristics: a weight of about 1000±50 g/m², a moisture of about 8%, a breaking load MD of about 175 N per 15 mm, a water capillary rise MD of about 100 mm per 10 minutes, and a pH board surface of about 7.0. Any combination of properties which provides for good absorption of the formulation and which allow, when heated, the formulation to be effectively evaporated, may be used.

According to another embodiment, the formulation may be used in a liquid form in conjunction with a suitable dispensing container. According to this embodiment, a suitable dispenser can include a container whose opening is fixed with a ceramic wick. Pest and insect control products may include formulations comprising at least one spinosyn, and in one embodiment spinosad, and a solvent filled in a container with an opening fixed with a ceramic wick thereby resulting in the evaporation of the formulation. According to one embodiment, the wick is heated by a diffuser. The properties of the wick, including porosity, may affect the rate at which the formulation evaporates. The wick is not limited to a ceramic wick and any art recognized wick may be used, e.g., plastic, paper, cord or carbon. In one embodiment of the invention, ceramic wicks, for example, amorphous silica wicks, have been found to be suitable for use with the present invention. The selection of an appropriate wick would be readily apparent to the skilled artisan based upon the formulation to be applied. Properties of the wick that one might consider include porosity, density, flexural strength and thermal conductivity. According to one embodiment, the wick for use with the present invention has a maximum porosity of about 55%, a density of more than about 1.0 g/cc, a flexural strength of more than about 0.4 Mpa, and a thermal conductivity of about 0.3 W/mK (20° C.-100° C.). Although these wick properties have been found to be suitable for use in the present invention, any properties which provide for a wick which allows for effective evaporation of the spinosyn formulation may be used.

Other recognized forms for the product include but are not limited to coils, candles, fumigators, and anti-moth products. Coils are generally formulated from ingredients including, but not limited to one or more powders, for example, wood powder and sticky powder, one or more starches, one or more colorants, one or more perfumes and one or more solvents and the active ingredient(s). In coils, burning compressed powder generates the heat required to vaporize the active(s).

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. It will be apparent to those skilled in the art that embodiments described herein may be modified or revised in various ways without departing from the spirit and scope of the invention.

### EXAMPLE 1

In a non transparent conical container, 20 grams of TRACER (or LASER) 48% (equivalent to 9.6 grams of pure spinosad) were mixed with 80 grams of ethyl alcohol. The mixture was stirred for three hours with a mechanical stirrer of the type IKA RCT at a speed of 500-800 RPM. The mixture was then stored at room temperature, without stirring, for 24 hours until it separated into two phases, the upper phase being an almost transparent liquid and the bottom phase containing almost all of the solids. The upper phase was carefully transferred in another container and was then analyzed by HPLC to confirm the concentration of spinosad A and D.

Paper mats having dimensions of 35 mm (length)×21.5 mm (width)×2.5 mm (thickness) were then gradually impregnated with 0.84-1.70 grams of the above upper phase mixture (depending on its content in spinosad) so as to have approximately 80 mgs of pure spinosad per mat. The mats were left at a temperature of approximately 30° C. for 3 hours during which time most of the ethyl alcohol was evaporated. Next the mats were closed within foil comprising metallized polyethylene terepthalate PET 12 μm/adhesive/PET 35 μm. The mats were allowed to rest for 24 hours to allow for complete spread of the liquid material within the paper. Evaporation tests were then run as follows:

The foil was opened and a mat was removed. The mat was checked by HPLC to confirm its spinosad content. As was theoretically expected, 80 mgs of pure spinosad per mat were found. Four mats of this batch were then placed on common mat diffusers having a temperature ranging from 130° C. to 160° C. The amount of spinosad remaining on the mat was checked after each of 2, 4, and 8 hours of diffuser operation. After 8 hours of operation, it was found that 75% to 98% of the spinosad had been evaporated from the mat (depending on the diffuser temperature)

| | Diffuser | Diffuser | Diffuser |
|---|---|---|---|
| Initial Quantity of Spinosad | Temperature | Temperature | Temperature |
| per mat: 80 mgs | 130° C. | 140° C. | 160° C. |
| Quantity of Spinosad | 20 | 12 | 2 |
| Remained on mat after 8 h | | | |
| of operation (mgs) | | | |
| Percentage of Spinosad | 75% | 85% | 98% |
| Evaporated | | | |

Sample mats from this batch were then sent to a biological test center in order to analyze their control and efficacy as against adult mosquitoes and it was found that after 6 hours of mat operation in a 20 cubic meter chamber an 85% 24 h-mortality was achieved.

### EXAMPLE 2

In a conical container, 5 grams of Tracer 44.2% (equivalent to 2.2 grams of pure spinosad) were mixed with 95 grams of ethylene glycol phenyl ether. The mixture was stirred for one hour with a mechanical stirrer of the type IKA RCT at a speed of 500-800 RPM until the complete solubility of spinosad was achieved. The mixture was then stored at room temperature for an additional hour within a non transparent container to avoid any possible photodegradation. The mixture was then analyzed by HPLC to confirm the concentration of spinosad A and D in the final mixture. Paper mats of dimensions 35 mm (length)×21.5 mm (width)×2.5 mm (thickness) were then impregnated with 0.5 grams of the above mixture. Next the mats were closed within a foil comprising metallized polyethylene terepthalate PET 12 μm/adhesive/PET 35 μm. The mats were allowed to sit for 24 hours in order to allow for a complete spread of the mixture within the paper. Evaporation tests were then run as follows:

The foil was opened and a mat was removed. The mat was checked by HPLC to confirm the content of spinosad. As was theoretically expected, 11 mgs of pure spinosad per mat were found. Four mats were then placed on common mat diffusers having a temperature ranging from 130° C. to 160° C. The amount of spinosad remaining on the mat was checked after each of 2, 4, and 8 hours of operation. After 8 hours of operation, it was found that 75% to 98% of the spinosad had been evaporated from the mat (depending on the diffuser temperature).

Sample mats from this batch were then sent to a biological test center in order to analyze their control and efficacy as against adult mosquitoes. It was found that after 4 hours of mat operation in a 1 cubic meter chamber, a KT95 of 27 min, a 100% knockdown after 2 hours and a 100% 24 h-mortality was achieved.

The same test was also carried out using methyl isobutyl ketone as a solvent with similar evaporation results for spinosad. The biological efficacy tests revealed a slightly lower knockdown rate with methyl isobutyl ketone (76% knockdown after 2 hours and a 100% 24 h-mortality).

The same mats were also tested against houseflies and were also proven to be effective for their control.

### EXAMPLE 3

In a conical container, 2.2 grams of spinosad technical (technical referring to spinosad, as produced, and including non-interfering impurities—the impurity amounts are provided by the manufacturer) (equal to 2 grams of pure spinosad) was mixed with 97.8 grams of ethylene glycol phenyl ether. The mixture was stirred for one hour with a mechanical stirrer of the type IKA RCT at a speed of 500-800 RPM until the complete solubility of spinosad was achieved. The mixture was then stored at room temperature for five days. After five days, no precipitate was observed. The mixture was then analyzed by HPLC to confirm the concentration of spinosad A and D in the final mixture. Paper mats of dimensions 35 mm (length)×21.5 mm (width)×2.5 mm (thickness) were then impregnated with 0.5 grams of the above mixture. Next the mats were closed within a foil comprising metallized polyethylene terepthalate PET 12 μm/adhesive/PET 35 μm. The mats were allowed to sit for 24 hours in order to allow for a complete spread of the mixture within the paper. Evaporation tests were then run as follows:

The foil was opened and a mat was removed. The mat was checked by HPLC to confirm the content of spinosad. As was theoretically expected, 10 mgs of pure spinosad per mat was found. Four mats were then placed on common mat diffusers having a temperature ranging from 130° C. to 160° C. The amount of spinosad remaining on the mat was checked after each of 2, 4, and 8 hours of operation. After 8 hours of operation, it was found that 75% to 98% of the spinosad had been evaporated from the mat (depending on the diffuser temperature).

Sample mats from this batch were then sent to a biological test center in order to analyze their efficacy for the control of adult mosquitoes. The results showed that, after 6 hours of mat operation as described above, 85% of the mosquitoes were knocked down and 75% (24 h) were killed. The same mats were also tested against houseflies and were also proven to be effective for their control.

### EXAMPLE 4

In a conical container, 4.2 grams of TRACER 48% (or LASER), equal to 2 grams of pure spinosad, or 2.2 grams of spinosad technical (equal to 2 grams of pure spinosad) were mixed with 2.2 grams of piperonyl butoxide and 93.6 grams (or 95.6 grams, respectively) of ethylene glycol phenyl ether. The mixture was stirred for one hour with a mechanical stirrer of the type IKA RCT at a speed of 500-800 RPM until the complete solubility of spinosad was achieved. The mixture was then stored at room temperature and away from the direct sunlight for five days. After five days, no precipitate was observed. The mixture was then analyzed by HPLC to confirm the concentration of spinosad A and D in the final mixture.

PET plastic bottles were then each filled with 24 grams of the above formulation. A plastic insert and ceramic wick were then placed in the opening of the bottle and the bottle was screwed onto a common diffuser. The diffuser was operated at a temperature ranging from 125°-150° C. After 8 hours, it was found that 1 gram of the mixture (equal to 20 mgs of pure spinosad) had been evaporated. The remainder of the mixture in the bottle was then analyzed by HPLC. This analysis indicated that the concentration of the remaining mixture had not changed. The bottle was then plugged off and stored for 12 additional hours. The bottle was again screwed onto a common diffuser, and the diffuser was operated at a temperature of 125°-150° C. The results after an additional 8 hours were similar to the results after the first 8 hours.

Selected samples of the bottles were then sent to a biological test center to be tested against adult mosquitoes and houseflies and were proven to be effective for their control.

1. A vaporizable pest control product comprising:
at least one spinosyn or derivative thereof; and
at least one vehicle to cause evaporation.

2. The product claim 1, wherein the at least one spinosyn is chosen from spinosyn A and spinosyn D.

3. The product of claim 1, wherein the vehicle is chosen from one or more of heat, airflow, pressure and at least one solvent.

4. The product of claim 2, wherein the at least one spinosyn is spinosad.

5. The product of claim 4, wherein the vehicle is heat.

6. The product of claim 5, wherein the vehicle further includes at least one solvent.

7. The product of claim 6, wherein the solvent comprises at least one of ethylene glycol phenyl ether and water.

8. The product of claim 7, wherein the amount of solvent is an amount sufficient to substantially dissolve the spinosyn.

9. The product of claim 1, further comprising at least one secondary active ingredient.

10. The product of claim 9, wherein the at least one secondary active ingredient comprises piperonyl butoxide.

11. A method for controlling pests comprising:
vaporizing at least one spinosyn or derivative thereof; and
contacting the pests with the vapor-phase spinosyn.

12. The method according to claim 11, wherein the at least one spinosyn is spinosad.

13. The method according to claim 12, further comprising, prior to vaporizing the spinosad, dissolving the spinosad in a solvent.

14. The method according to claim 13, wherein the solvent comprises ethylene glycol phenyl ether or ethyl alcohol.

15. The method according to claim 14, wherein the amount of solvent is an amount sufficient to substantially dissolve the amount of spinosad.

16. The method according to claim 13, further comprising, prior to vaporizing the spinosad, applying the solution of spinosad and solvent to a storage medium.

17. The method according to claim 16, wherein the storage medium is a mat comprising paper, plastic, fabric, or ceramic.

18. The method according to claim 16, wherein the storage medium is a coil.

19. The method according to claim 16, wherein the storage medium is a container comprising a wick.

20. The method according to claim 19, wherein the wick made from one or more materials chosen from ceramic, plastic, paper, cord, and carbon.

21. The method according to claim 20, wherein the ceramic wick is amorphous silica.

22. A pest control product capable of producing a vaporized insecticide comprising:
at least one spinosyn or derivative thereof dissolved in a solvent;
a paper mat capable of absorbing the at least one spinosyn or derivative thereof dissolved in the solvent; and
a diffuser capable of applying heat to the paper mat in order to vaporize the at least one spinosyn dissolved in the solvent.

23. The product according to claim 22, wherein the solvent is chosen from one or more of water, methyl isobutyl ketone, ethyl alcohol and ethylene glycol phenyl ether.

24. The product according to claim 23, wherein the amount of solvent is an amount sufficient to substantially dissolve the spinosyn.

25. A pest control product capable of producing a vaporized insecticide comprising:
at least one spinosyn or a derivative thereof dissolved in a solvent;
a container capable of storing the at least one spinosyn or derivative thereof dissolved in the solvent;
a wick attached to the container; and
a diffuser capable of applying heat to the wick in order to vaporize the at least one spinosyn dissolved in the solvent.

26. The product according to claim 25, wherein the solvent comprises ethylene glycol phenyl ether.

27. The product according to claim 26, wherein the solvent is in an amount sufficient to substantially dissolve the at least one spinosyn.

28. A method of controlling adult mosquitoes comprising
vaporizing at least one spinosyn or derivative thereof,
contacting said adult mosquitoes with said at least one vapor-phase spinosyn or derivative thereof.

29. The method according to claim 28, wherein said spinosyn is spinosad.

30. The method according to claim 29, wherein said spinosad is dissolved in ethylene glycol phenyl ether or ethyl alcohol to form a solution.

31. The method according to claim 30, wherein said solution is heated.

32. A method of controlling houseflies comprising
vaporizing at least one spinosyn or derivative thereof,
contacting said houseflies with said at least one vapor-phase spinosyn or derivative thereof.

33. The method according to claim 32, wherein said spinosyn is spinosad.

34. The method according to claim 33, wherein said spinosad is dissolved in ethylene glycol phenyl ether or ethyl alcohol to form a solution.

35. The method according to claim 34, wherein said solution is heated.

36. A method of controlling pests in stored grain comprising:
vaporizing at least one spinosyn or derivative thereof; and
contacting the stored grain with the vapor-phase spinosyn.

37. The method according to claim 36, wherein the at least one spinosyn is spinosad.

38. The method according to claim 37, further comprising, prior to vaporizing the spinosad, dissolving the spinosad in a solvent.

39. The method according to claim 38, wherein the solvent comprises ethylene glycol phenyl ether or ethyl alcohol.

40. The method according to claim 39, wherein the amount of solvent is an amount sufficient to substantially dissolve the amount of spinosad.

41. The product of claim 1, wherein the spinosyn comprises a mixture of (2R,3aR,5aR,5bS,9S,13S,14R,16aS,16bR)-13-{[(2R,5S,6R)-5-(dimethylamino)-6-methyltetrahydro-2H-pyran-2-yl]oxy}-9-ethyl-14-methyl-7,15-dioxo-2,3,3a,4-,5,5a,5b,6,7,9,10,11,12,13,14,15,16a,16b-octadecahydro-1H-as-indaceno[3,2-d]oxacyclododecin-2-yl 6-deoxy-3-O-ethyl-2,4-di-O-methyl-.alpha.-L-mannopyranoside and (2S,3aR,5aS,5bS,9S,13S,14R,16aS,16bS)-13-{[(2R,5S,6R)-5-(dimethylamino)-6-methyltetrahydro-2H-pyran-2-yl]oxy}-9-ethyl-4,14-dimethyl-7,15-dioxo-2,3,-3a,5a,5b,6,7,9,10,11,12,13,14,15,16a,16b-hexadecahydro-1H-as-indaceno[3,2-d]oxacyclododecin-2-yl 6-deoxy-3-O-ethyl-2,4-di-O-methyl-.alpha.-L-mannopyranoside in the proportion 50-90% to 50-10%

42. A pest control method which comprises dispersing in a space where pest control is desired a composition comprising spinosyn and a liquid carrier, wherein said composition is dispersed to produce a fog.

43. The method according to claim 42, wherein the composition comprises at least one of spinosad and spinetoram.

44. The method according to claim 42 wherein said composition is dispersed using a thermal fogger.

45. The method according to claim 42, wherein said composition is dispersed in an enclosed space.

46. The method according to claim 42, used to control at least one of beetles, cockroaches, and ants.

47. The method according to claim 42, used to control at least one of flies and moths.

48. The method according to claim 42, wherein said space is in a greenhouse.

49. The method according claim 45, wherein said enclosed space contains plant material it is desired to protect from pests.

50. The method according to claim 45, wherein said enclosed space contains stored products it is desired to protect from pests.

51. A method for disinfecting a space which comprises dispersing in said space a composition comprising a spinosyn and a non-aqueous organic solvent in the form of a fog.

52. An electric mosquito control mat comprising a substrate impregnated with spinosyn.

53. The electric mosquito control mat according to claim 52, wherein the spinosyn is spinetoram.

54. A combustible coil comprising a combustible carrier and a spinosyn.

55. The combustible coil according to claim 54, wherein the spinosyn is spinetoram.

56. A pest control method which comprises dispersing in a space where pest control is desired a composition comprising a spinosyn and a liquid carrier, wherein said composition is dispersed in the form of a vapor.

57. The pest control method according to claim 56, wherein the spinosyn is spinetoram.

58. The method according to claim 56, wherein said composition is dispersed in an enclosed space.

59. A method for disinfecting a space which comprises dispersing in said space a composition comprising a spinosyn and a non-aqueous organic solvent in the form of a vapor.

60. The method according to claim 59, wherein the spinosyn is spinetoram.
